(19) United States
(12) Patent Application Publication
(10) Pub. No.: US 2022/0244182 A1
(43) Pub. Date: Aug. 4, 2022

(54) METHODS FOR IDENTIFYING NUCLEIC ACIDS IN A SAMPLE

(71) Applicant: LexaGene, Inc., Beverly, MA (US)

(72) Inventors: Nathan Walsh, Westborough, MA (US); Jacob Silterra, Malden, MA (US)

(73) Assignee: LexaGene, Inc., Beverly, MA (US)

(21) Appl. No.: 17/607,180

(22) PCT Filed: Apr. 22, 2020
(86) PCT No.: PCT/US2020/029199

§ 371 Date: Oct. 28, 2021

Related U.S. Application Data
(60) Provisional application No. 62/839,845, filed on Apr. 29, 2019.

(51) Int. Cl. G01N 21/64; G01N 1/28; C12Q 1/6895; C12Q 1/689

(52) U.S. Cl. CPC G01N 21/6456; G01N 21/6428; C12Q 1/686; C12Q 1/6895; C12Q 1/689; G01N 1/28

(57) ABSTRACT

The invention includes methods for identifying nucleic acids in a sample. The invention provides methods for tracking reaction mixtures in a defined flow path so that nucleic acids in the reaction mixtures can be identified. The invention provides methods for calculating the movement of reaction mixtures in a defined flow path, including defined flow paths that are nonlinear and/or have complicated shapes.

### CROSS-REFERENCE TO RELATED APPLICATIONS

This application claims priority to U.S. Provisional Patent Application No. 62/839,845 filed Apr. 29, 2019. The foregoing application is hereby incorporated by reference in its entirety.

### FEDERALLY SPONSORED RESEARCH OR DEVELOPMENT

Not Applicable.

### JOINT RESEARCH AGREEMENT

Not applicable.

### TECHNICAL FIELD

The invention generally relates to identification of a sample. The invention more specifically relates to optical identification of nucleic acids in a sample.

### BACKGROUND

In polymerase chain reaction (PCR)-based identification machines that image reaction mixtures in defined flow paths, such as tubes and/or channels, the reaction mixtures can move from image to image. The movement of the reaction mixtures in defined flow paths makes it difficult to track the reaction mixtures from image to image and therefore difficult to analyze the images. An inability to properly analyze the images can ultimately lead to an inability to identify nucleic acids in a sample.

Thus, there is a need for a means for identifying nucleic acids in a sample that overcomes the aforementioned problems and limitations.

### SUMMARY

The invention includes methods for identifying nucleic acids in a sample. The invention overcomes the aforementioned problems and limitations by providing a means for tracking reaction mixtures in a defined flow path so that nucleic acids in the reaction mixtures can be identified. The invention provides methods for calculating the movement of reaction mixtures in a defined flow path, including defined flow paths that are nonlinear and/or have complicated shapes, and thereby allowing for the identification of nucleic acids in the reaction mixtures.

Unless otherwise defined, all terms used herein have the same meaning as commonly understood by a person having ordinary skill in the art to which the invention pertains. All patents, patent applications, publications, and other references mentioned herein and/or listed in the Application Data Sheet are hereby incorporated by reference in their entirety. In case of conflict, the specification will control. When a range of values is provided, the range includes the end values.

The materials, methods, components, features, embodiments, examples, and drawings disclosed herein are illustrative only and not intended to be limiting.

### DESCRIPTION OF DRAWINGS

The invention is best understood from the following detailed description when read in connection with the drawings disclosed herein, with similar elements having the same reference numbers. When a plurality of similar elements is present, a single reference number may be assigned to the plurality of similar elements with a small letter designation referring to at least one specific similar element. When referring to the similar elements collectively or to a non-specific similar element, the small letter designation may be dropped. The various features of the drawings may not be drawn to scale and may be arbitrarily expanded or reduced for clarity. Included in the drawings are the following figures:

FIG. 1 is a diagram depicting an imager.

FIG. 2 is a diagram depicting a defined flow path.

FIG. 3 is a diagram depicting various defined flow paths.

FIG. 4 is a diagram depicting a segment of a defined flow path.

FIG. 5 is a diagram depicting multiple images of a segment of a defined flow path.

FIG. 6 is a flow chart of steps for identifying nucleic acids in a sample.

FIG. 7 is a diagram depicting a linear representation of a nonlinear defined flow path.

### DEFINITIONS

To facilitate understanding of the invention, a number of terms are defined herein.

A “defined flow path” is the path that a fluid (liquid and/or gas) can travel. The defined flow path can be a tube, channel, or any other defined path. In some embodiments, the channel is a microfluidic channel etched in glass or plastic or a tube that is made of plastic and is rigid or flexible. The defined flow path can have any shape including but not limited to a shape that is linear, curved, spiral, or serpentine. The defined flow path can also have one or more of the aforementioned shapes. For example, in some embodiments, the defined flow path will have a linear segment, followed by a serpentine segment, and then followed by another linear segment. The segments can be the same material or different materials. For example, the linear segments can be made of a rigid tube and the serpentine segment can be made of a flexible tube. The defined flow path can also have shapes that are not easily described.

A “fluid” is a liquid and/or gas.

A “fluorescent compound” is a probe that fluoresces when excited under a specific set of conditions. Examples of a specific set of conditions include but are not limited to the presence of a nucleic acid sequence, the absence of a nucleic acid sequence, and a change in the relative concentrations of a nucleic acid sequence and at least one other nucleic acid sequence.

A “nucleic acid” is a biopolymer comprised of at least one nitrogenous base, commonly adenine, cytosine, guanine, thymine, and uracil; at least one sugar, commonly a 5-carbon sugar; and optionally at least one phosphate group. A nucleic acid can be of natural or artificial origin (artificial construct), or a combination thereof. Common sources of nucleic acids are organisms including but not limited to bacteria, fungi, animals, insects, and plants, and non-organisms including but not limited to viruses. Examples of sources of nucleic acids include but are not limited to organisms of the species Escherichia coli and/or the genera Streptococcus, Staphylococcus, Enterococcus, Klebsiella, Proteus, Acinetobacter, Pseudomonas, Enterobacter, Salmonella, Campylobacter, Chronobacter, Listeria, Botrytis and Sclerotinia.

A “property” is a measure of fluorescence or a calculation derived from the measurement of fluorescence. A property can relate to a fluid in an image (“fluid property”) or a fluid across the images of an image set (“curve property”).

A “reaction mixture” is a fluid that contains PCR reagents including but not limited to at least one fluorescent compound, primers, nucleotides, and an enzyme.

A “sample” is an input that may contain one or more nucleic acids.

### DETAILED DESCRIPTION

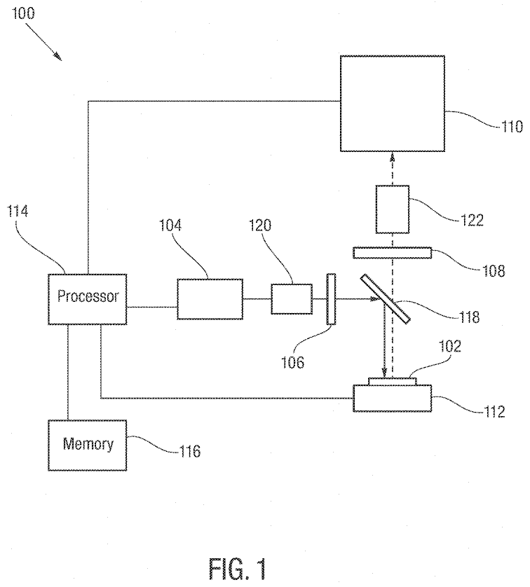

FIG. 1 depicts an imager in accordance with aspects of the invention. The imager 100 includes a defined flow path 102, an illumination source 104, an illumination filter 106, a camera filter 108, a camera 110, a thermal device 112, a processor 114, and a memory 116. In this nonlimiting embodiment, the imager also includes a dichroic mirror 118, an illumination lens 120, and a camera lens 122. The illumination source 104 generates illumination light that travels through the illumination filter 106 and illuminates the defined flow path 102. In this nonlimiting embodiment, the illumination light (also referred to as “excitation light”) travels through the illumination lens 120 and the illumination filter 106, is redirected by the dichroic mirror 118, and then illuminates the defined flow path 102. If the illumination light excites one or more fluorescent compounds in the defined flow path, then the one or more fluorescent compounds generate emission light which travels through the dichroic mirror 118 and the camera filter 108 and enters the camera 110 where an image is captured. In this embodiment, the emission light from the excited one or more fluorescent compounds travels through the dichroic mirror 118, the camera filter 108, the camera lens 122, and then enters the camera 110. The processor 114 is in operable connectivity with the camera 110, the illumination source 104, the thermal device 112, and the memory 116. In a preferred embodiment, three illumination filters and three camera filters are arranged in pairs such that a specific illumination filter is used with a specific camera filter. The pairs may be physically fixed, such as in a linear box array, or unfixed and therefore independently positionable, such as in two independent filter wheels. The illumination source 104 can be any device that emits light including but not limited to a laser, LED, filament, or halogen light source. In some embodiments, multiple illumination sources are used. In some embodiments, 0, 1, 2, 3, 4, 5, 6, 7, 8, 9, 10, 11, 12, 13, 14, 15, 16, 17, 18, 19, 20, 25, 30, 35, 40, 45, 50 or more illumination filters are used with each illumination source. The camera 110 can be any optical device that captures an image including but not limited to a charge-coupled device (CCD), photodiode, and photomultiplier tube. At least one camera filter is used to filter the light entering a camera. In some embodiments, multiple cameras are used. In some embodiments, 1, 2, 3, 4, 5, 6, 7, 8, 9, 10, 11, 12, 13, 14, 15, 16, 17, 18, 19, 20, 25, 30, 35, 40, 45, 50, or more camera filters are used with each camera.

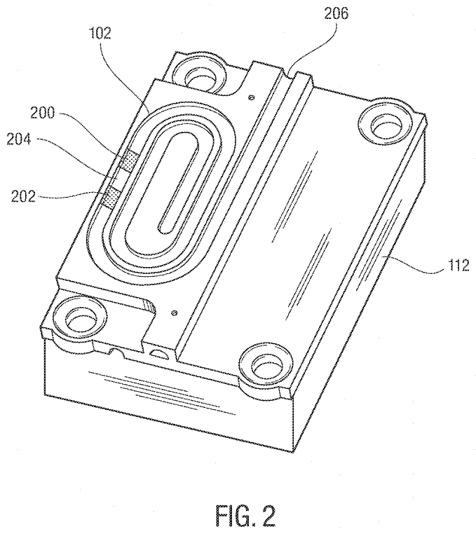

FIG. 2 depicts the defined flow path 102 containing a first fluid 200, a second fluid 202, and a third fluid 204. In this embodiment, the thermal device 112 is adjacent to the defined flow path 102, but the thermal device 112 may be in any position relative to and at any distance from the defined flow path 102 that allows for thermal connectivity with the defined flow path 102. The thermal device 112 controls the temperature of the first fluid 200, the second fluid 202, and the third fluid 204 (collectively, “fluids”) in the defined flow path 102. The thermal device 112 will be in operable connectivity with a processor (not depicted) and can maintain a temperature and/or cycle between two or more temperatures. The thermal device 112 controls the temperature of the fluids by heating and/or cooling the fluids. The thermal device can use any combination of active heating, passive heating, active cooling, and passive cooling to control the temperature of the fluids. In some embodiments, the thermal device 112 is next to the defined flow path 102 such that the thermal device 112 interfaces with the defined flow path 102 in two dimensions. In other embodiments, the defined flow path 102 is partially of fully embedded in the thermal device 112 such that the thermal device interfaces with the defined flow path 102 in three dimensions. Examples of a thermal device include but are not limited to a Peltier device, radiation device, and heat pump. The interface between two fluids can be any shape including but not limited to linear or curved, such as concave or convex. For example, in a hydrophobic tube, the interface between an aqueous fluid and oil is typically curved with the aqueous fluid exhibiting a convex shape.

The first fluid 200, the second fluid 202, and the third fluid 204 can each be a liquid and/or gas. The fluids enter the defined flow path through an inlet 206 and, once in the defined flow path 102, can travel away from or towards the inlet 206. The defined flow path 102 can also have an optional outlet. As depicted in this figure, the defined flow path 102 can have more than one fluid in it simultaneously. In some embodiments, the defined flow path has 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 100, 200, 300, 400, 500, 1000, or more fluids in the defined flow path simultaneously. In some embodiments, the fluids alternate between aqueous and gas, and, in preferred embodiments, the fluids alternate between aqueous and oil. In a preferred embodiment, the number of fluids in the defined flow path is between 20 and 40. In this embodiment, the first fluid 200 is aqueous, the second fluid 202 is aqueous, and the third fluid 204 is oil.

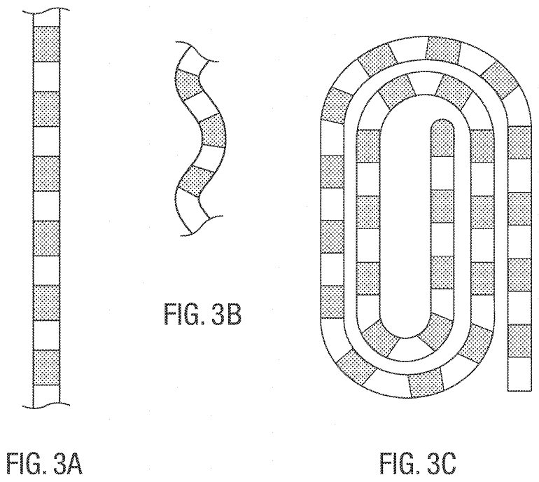

FIG. 3 depicts various defined flow paths in accordance with aspects of the invention. FIG. 3A depicts a linear defined flow path with multiple fluids. FIG. 3B depicts a winding defined flow path with multiple fluids. FIG. 3C depicts a spiral defined flow path with multiple fluids.

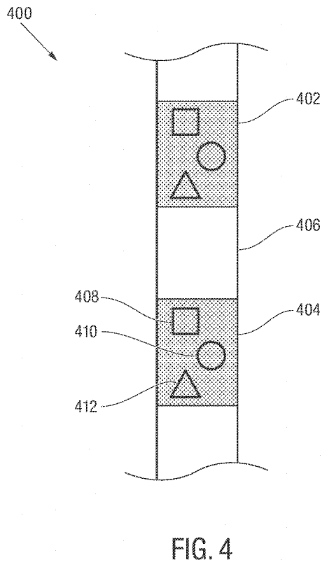

FIG. 4 depicts a linear segment of a defined flow path 400 containing three fluids. In this embodiment, a first fluid 402 and a second fluid 404 are aqueous reaction mixtures containing PCR reagents including a first fluorescent compound 408 (depicted by a square), a second fluorescent compound 410 (depicted by a circle), and a third fluorescent compound 412 (depicted by a triangle). A third fluid 406 separates the first fluid 402 and the second fluid 404, and the third fluid is not a reaction mixture. In a preferred embodiment, the third fluid is oil. Each fluorescent compound is a probe that fluoresces under a specific set of conditions. One example of conditions when a fluorescent compound will fluoresce is when it binds to a particular nucleic acid sequence. For example, TaqMan® probes (Thermo Fisher Scientific, Waltham, Mass., USA) use a probe linked to a fluorophore and a quencher. When the probe is not bound to a particular nucleic acid sequence, the quencher is able to quench the fluorophore and the fluorophore will not fluoresce. However, when the probe binds to a particular nucleic acid, an enzyme, such as Taq polymerase, cleaves between the quencher and the fluorophore and as a result the quencher is not able to quench the fluorophore and the fluorophore will fluoresce. Whether the fluorophore fluoresces indicates whether the probe bound to a particular nucleic acid and therefore indicates whether the particular nucleic acid was present. In the embodiment of FIG. 4, each of the three fluorescent compounds selectively binds to a different nucleic acid. For example, the fluorophore of the first fluorescent compound can be FAM® (Thermo Fisher Scientific, Waltham, Mass., USA), the fluorophore of the second fluorescent compound can be HEX® (Thermo Fisher Scientific, Waltham, Mass., USA), and the fluorophore of the third fluorescent compound can be Cy®5 (GE Healthcare, Amersham, Chicago, Ill., USA). Each of three fluorescent compounds produces a different excitation band of wavelengths. As is commonly understood in the art, using different illumination filters and camera filters allows for the imaging of a single fluorescent compound at a time. The advantage of this method is that more than one fluorescent compound can be in a fluid containing more than one nucleic acid and each nucleic acid can be identified.

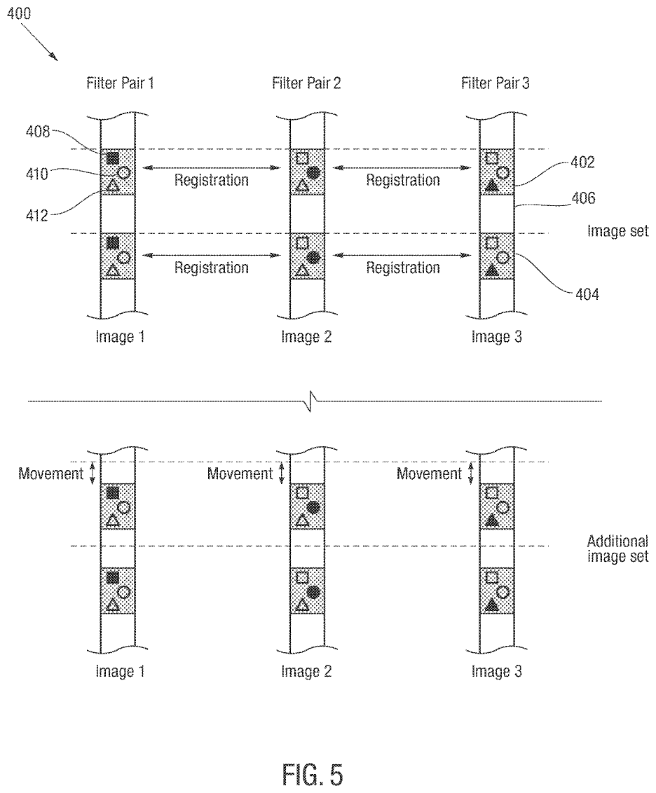

FIG. 5 depicts six images (two image sets of three images each) of the linear segment of defined flow path of FIG. 4. In the top-left image, the first fluorescent compound 408 is fluorescing, as depicted by a solid square. In the top-middle image, the second fluorescent compound 410 is fluorescing, as depicted by a solid circle. In the top-right image, the third fluorescent compound 412 is fluorescing, as depicted by a solid triangle. The three images on the top row were captured sequentially and are part of the same image set. In the bottom-left image, the first fluorescent compound 408 is fluorescing. In the bottom-middle image, the second fluorescent compound 410 is fluorescing. In the bottom-right image, the third fluorescent compound 412 is fluorescing. The three images on the bottom row were captured sequentially and are part of the same image set (an additional image set). The first image set was created first and at a later time the additional image set was created. The figure illustrates that each fluid was registered across each image, which is described in greater detail below. In this embodiment, only the first fluid 402 and the second fluid 404 are registered because the third fluid 406 is not a reaction mixture. The figure also illustrates the movement of the fluids in the defined flow path that occurred between the creation of the image set and the creation of the additional image set. Movement of fluids in a defined flow path is described in greater detail below.

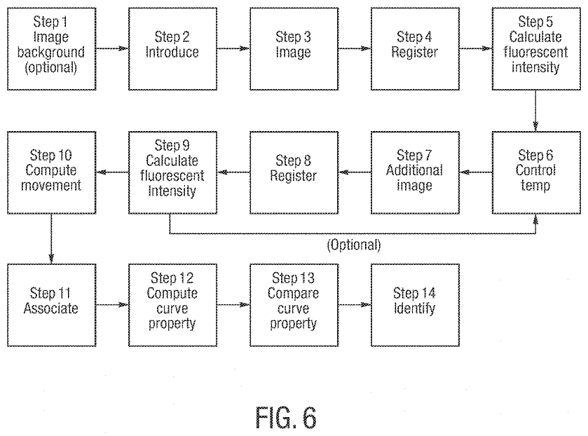

FIG. 6 depicts a flow chart of a preferred embodiment for identifying at least one nucleic acid in a defined flow path. In a preferred embodiment, three images are captured each time the defined flow path is imaged (to create an image set of three images). In a preferred embodiment, three pairs of illumination filters and camera filters are provided, and each filter pair is used one at a time with a particular fluorescent compound. In a preferred embodiment, three fluorescent compounds are in each fluid that is a reaction mixture and each fluorescent compound is imaged using a different filter pair. For illustrative purposes only, three nucleic acids will be assumed.

In step 1, which is an optional step, three images of the defined flow path are captured before at least one fluid is introduced to the defined flow path. The image, referred to as a “background image,” is an image of the defined flow path and anything else that is in the imagable field of view including but not limited to a substance in or on the defined flow path such as dust, an imperfection in or on the defined flow path, and any autofluorescence. In a preferred embodiment and for illustrative purposes only, step 1 is performed and three background images are captured.

In step 2, three fluids are introduced to the defined flow path. Any number of fluids can be introduced, and three fluids are used for illustrative purposes only. The first fluid and the second fluid are reaction mixtures containing PCR reagents including but not limited to three fluorescent compounds, primers, nucleotides, and an enzyme. In this example, the first fluid and the second fluid also contain a sample that includes three nucleic acids. The third fluid separates the first fluid and the second fluid, and it is not a reaction mixture.

In step 3, three images of the defined flow path, which now contains the first fluid and the second fluid (separated by the third fluid), are captured. The three background images from step 1 are subtracted from the three images. A background image captured using a specific filter pair is subtracted from an image captured using the same specific filter pair. Therefore, the subtraction occurs three times to create an image set containing three images.

In step 4, the first fluid and the second fluid are registered across the image set, meaning that the first fluid and the second fluid are registered in each image (in this example, three images) of the image set. The first fluid is registered by calculating at least one fluid property of the first fluid and grouping the first fluid across the image set based on the at least one fluid property. The at least one fluid property of the first fluid includes but is not limited to the fluorescent intensity, size, and position of the first fluid. Fluorescent intensity can be calculated by a number of methods including but not limited to averaging the fluorescent intensity of each pixel, selecting the maximum intensity of a pixel, using a particular percentile of the rank order of the fluorescent intensity of the pixels, or it can be read directly from the output of the camera. Size can be calculated by a number of methods including but not limited to counting the number of pixels in the fluid or a calculation that accounts for the shape of the defined flow path, for example calculating a three-dimensional volume. Position can be calculated by a number of means including but not limited to starting with the location of a pixel, for example the x and y coordinates of a pixel in the center of the fluid (also referred to as a “centroid”), and calculating the mathematical transformation of the x and y coordinates of the centroid. Some mathematical transformations of nonlinear defined flow paths are further described below. The second fluid is registered by calculating at least one fluid property of the second fluid and grouping the second fluid across the image set based on the at least one fluid property of the second fluid. The at least one fluid property includes but is not limited to the fluorescent intensity, size, and position of the second fluid. The at least one fluid property of the first fluid and the second fluid do not need to be the same nor do the number of fluid properties calculated for each fluid. A person skilled in the art will understand that other fluid properties may be used to register the first fluid and the second fluid across the image set.

In step 5, the fluorescent intensity of the first fluid and the second fluid is calculated in at least one image of the image set. If the fluorescent intensity was already calculated for the first fluid and/or the second fluid in step 4, then step 5 is not performed for the first fluid and/or the second fluid. In addition, optionally, at least one of the size and position of the first fluid and the second fluid is calculated across the image set.

In step 6, the temperature of the first fluid and the second fluid is controlled using a thermal device to amplify the three nucleic acids in the first fluid and the second fluid by PCR.

In step 7, three additional images of the defined flow path are captured and the three background images from step 1 are subtracted from the three images in the same manner as step 3 to create an additional image set containing three images.

In step 8, the first fluid and the second fluid are registered across the additional image set in the same manner as step 4.

In step 9, the fluorescent intensity of the first fluid and the second fluid is calculated in at least one image of the additional image set. If the fluorescent intensity was already calculated for the first fluid and/or the second fluid in step 8, then step 9 is not performed for the first fluid and/or the second fluid.

The sequence of steps 6, 7, 8, and 9 is then optionally repeated at least one time to create at least one additional image set. In some embodiments, the sequence of steps 6, 7, 8, and 9 is repeated 10 to 40 times to create 10 to 40 additional image sets. In preferred embodiments, the sequence of steps 6, 7, 8, and 9 is repeated 38 to 40 times to create 38 to 40 additional image sets.

In step 10, the movement of the first fluid and the second fluid in the defined flow path is computed. Because the image sets are created at different times, the fluids may have moved from image set to image set. The fluids may have moved due to the heating and/or cooling of the fluids by the thermal device. For example, if the thermal device cycles the fluids between a higher temperature and a lower temperature, preferably between a temperature above 75 degrees Celsius (° C.) and a temperature below 75° C., the fluids tend to move along the defined flow path as a result of expansion and/or contraction of the fluids and/or the defined flow path. This movement along the defined flow path is termed “drift” because the movement tends to occur steadily with each cycle of heating and/or cooling of the fluids. The first fluid and the second fluid can be tracked from image set to image set based on at least one fluid property of each fluid such as fluorescent intensity, size, and position. A person skilled in the art will understand that other fluid properties may be used to compute the movement of the first fluid and the second fluid from image set to image set.

In step 11, the first fluid and the second fluid are associated with the three nucleic acids to create three fluorescent intensity groups for the first fluid and the second fluid. In a preferred embodiment, one of the groups is an internal positive control. The association of the fluids can use any known information about the first fluid and the second fluid including but not limited to any fluid property and the order of the fluids (relative position of the fluids to one another) in the defined flow path.

In step 12, at least one curve property is computed for each of the three fluorescent intensity groups. In some embodiments, the at least one curve property is crossing threshold (Ct) and/or relative fluorescent units (RFU), and in a preferred embodiment both the Ct and RFU are calculated.

In step 13, the at least one curve property of each of the fluorescent intensity group is compared to a library of at least one curve property of known nucleic acids.

In step 14, the three nucleic acids in the sample are identified based on the results of step 13.

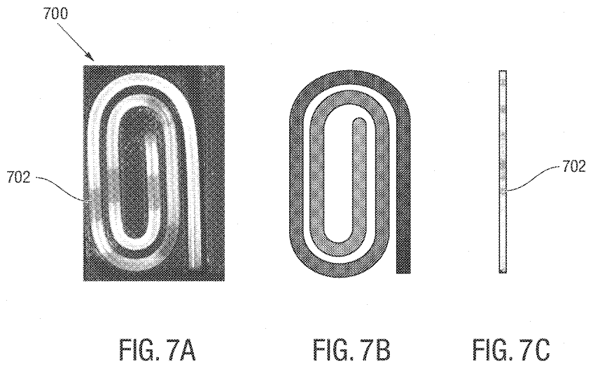

FIG. 7 depicts a linear representation of a nonlinear defined flow path. Calculating fluid properties, such as position, in a nonlinear defined flow path can be accomplished by transforming the nonlinear defined flow path to a linear representation, also referred to as a “linearized representation.” FIG. 7A is an image of a spiral defined flow path 700 containing multiple fluids including fluid 702 in a nonlinear state. FIG. 7B shows the same spiral defined flow path in a grayscale gradient where the color of the gradient changes from dark to light (or, alternatively, light to dark) as the distance from the input of the defined flow path increases. FIG. 7C is the linear representation of the image in FIG. 7A and fluid 702 is depicted in a linearized state. A person skilled in the art will understand that there are many methods to mathematically define distance, including but not limited to the Manhattan distance and the Chebyshev distance, and therefore there are many methods for transforming the nonlinear defined flow path to a linear representation.

What is claimed is:

1. A method for identifying at least one nucleic acid in a sample, comprising:
(a) providing an imager comprising a defined flow path, a thermal device in thermal connectivity with the defined flow path, a camera positioned to image the defined flow path, an illumination source positioned to illuminate the defined flow path, at least one camera filter positionable to filter light entering the camera, a memory, and a processor, wherein the processor is in operable connectivity with the camera, the illumination source, the thermal device, and the memory;
(b) optionally imaging the defined flow path by illuminating the defined flow path using the illumination source and capturing at least one background image of the defined flow path using the camera and the at least one camera filter;
(c) introducing the sample to the defined flow path in a first fluid wherein the first fluid further comprises polymerase chain reaction (PCR) reagents comprising at least one fluorescent compound and a second fluid wherein the second fluid further comprises polymerase chain reaction (PCR) reagents comprising at least one fluorescent compound and wherein the first fluid and the second fluid are separated by a third fluid;
(d) imaging the defined flow path by illuminating the defined flow path using the illumination source and capturing at least one image of the defined flow path using the camera and the at least one camera filter, and optionally subtracting the at least one background image, to create an image set;
(e) registering the first fluid and the second fluid across the image set if the image set has at least two images by calculating at least one fluid property of the first fluid and grouping the first fluid across the at least two images of the image set based on the at least one fluid property of the first fluid and calculating at least one fluid property of the second fluid and grouping the second fluid across the at least two images of the image set based on the at least one fluid property of the second fluid;
(f) calculating the fluorescent intensity of the first fluid and the second fluid in at least one image of the image set if the fluorescent intensity was not calculated in step (e) and optionally calculating at least one of the size and position of the first fluid and at least one of the size and position of the second fluid;
(g) controlling the temperature of the first fluid and the second fluid using the thermal device to amplify the at least one nucleic acid in the first fluid and the second fluid by polymerase chain reaction (PCR);
(h) imaging the defined flow path by illuminating the defined flow path using the illumination source and capturing at least one image of the defined flow path using the camera and the at least one camera filter, and optionally subtracting the at least one background image, to create an additional image set;
(i) registering the first fluid and the second fluid across the additional image set if the additional image set has at least two images by calculating at least one fluid property of the first fluid and grouping the first fluid across the at least two images of the additional image set based on the at least one fluid property of the first fluid and calculating at least one fluid property of the second fluid and grouping the second fluid across the at least two images of the additional image set based on the at least one fluid property of the second fluid;
(j) calculating the fluorescent intensity of the first fluid and the second fluid in at least one image of the additional image set if the fluorescent intensity was not calculated in step (i) and optionally calculating at least one of the size and position of the first fluid and at least one of the size and position of the second fluid;
(k) computing the movement of the first fluid and the second fluid in the defined flow path and applying a correction factor for the movement;
(l) associating the fluorescent intensity of the first fluid and the second fluid in each image set with the at least one nucleic acid to create a fluorescent intensity group for each at least one nucleic acid for the first fluid and second fluid;
(m) computing at least one curve property of the fluorescent intensity group for each at least one nucleic acid;
(n) comparing the at least one curve property to a library of the at least one curve property of known nucleic acids; and
(o) identifying the at least one nucleic acid in the sample.

2. The method for identifying at least one nucleic acid in a sample of claim 1, wherein step (g) is repeated at least one time.

3. The method for identifying at least one nucleic acid in a sample of claim 1, wherein the sequence of steps (h), (i), (j) is repeated at least one time.

4. The method for identifying at least one nucleic acid in a sample of claim 1, wherein the sequence of steps (g), (h), (i), and (j) is repeated at least one time.

5. The method for identifying at least one nucleic acid in a sample of claim 1, wherein step (g) controls the temperature of the first fluid and the second fluid by cycling the temperature of the first fluid and the second fluid between a temperature above 75 degrees Celsius (° C.) and a temperature below 75 degrees Celsius (° C.) using the thermal device.

6. The method for identifying at least one nucleic acid in a sample of claim 1, wherein the at least one fluid property of the first fluid of step (e) and step (i) is at least one of fluorescent intensity and position and the at least one fluid property of the second fluid of step (e) and step (i) is at least one of fluorescent intensity and position.

7. The method for identifying at least one nucleic acid in a sample of claim 1, wherein step (l) associates the fluorescent intensity of the first fluid in each image set with the at least one nucleic acid by using at least one of the size, fluorescent intensity, and position of the first fluid and the fluorescent intensity of the second fluid in each image set with the at least one nucleic acid by using at least one of the size, fluorescent intensity, and position of the second fluid.

8. The method for identifying at least one nucleic acid in a sample of claim 4, wherein the sequence of steps (g), (h), (i), and (j) is repeated 10 to 40 times.

9. The method for identifying at least one nucleic acid in a sample of claim 8, wherein the sequence of steps (g), (h), (i), and (j) is repeated 38 to 40 times.

10. The method for identifying at least one nucleic acid in a sample of claim 1, wherein the at least one curve property of the fluorescent intensity group of step (m) is at least one of crossing threshold (Ct) and relative fluorescent units (RFU).

11. The method for identifying at least one nucleic acid in a sample of claim 1, wherein the defined flow path contains at least one of a linear segment, a curved segment, a spiral segment, and a serpentine segment.

12. The method for identifying at least one nucleic acid in a sample of claim 1, wherein the first fluid is aqueous, the second fluid is aqueous, and the third fluid is at least one of oil and gas.

13. The method for identifying at least one nucleic acid in a sample of claim 1, wherein at least one illumination filter is provided and wherein the at least one illumination filter is positionable to filter light exiting the illumination source.

14. The method for identifying at least one nucleic acid in a sample of claim 1, wherein the at least one nucleic acid is selected from the group consisting of viruses, bacteria, fungi, animals, insects, plants, and artificial constructs.

15. The method for identifying at least one nucleic acid in a sample of claim 1, wherein the at least one nucleic acid is selected from the group consisting of the species Escherichia coli, and the genera Streptococcus, Staphylococcus, Enterococcus, Klebsiella, Proteus, Acinetobacter, Pseudomonas, Enterobacter, Salmonella, Campylobacter, Chronobacter, and Listeria.

16. The method for identifying at least one nucleic acid in a sample of claim 1, wherein the at least one nucleic acid is selected from the group consisting of the genera Botrytis or Sclerotinia.

17. The method for identifying at least one nucleic acid in a sample of claim 1, wherein the at least one nucleic acid is at least one of DNA and RNA.

18. A method for identifying at least one nucleic acid in a sample, comprising:
(a) providing an imager comprising a defined flow path, a thermal device in thermal connectivity with the defined flow path, a camera positioned to image the defined flow path, an illumination source positioned to illuminate the defined flow path, three camera filters positionable to filter light entering the camera, a memory, and a processor, wherein the processor is in operable connectivity with the camera, the illumination source, the thermal device, and the memory;
(b) imaging the defined flow path by illuminating the defined flow path using the illumination source and capturing three background images of the defined flow path using the camera and the three camera filters, wherein one camera filter is positioned to filter light entering the camera when a background image is captured;
(c) introducing the sample to the defined flow path in a first fluid wherein the first fluid further comprises polymerase chain reaction (PCR) reagents comprising three fluorescent compounds and a second fluid wherein the second fluid further comprises polymerase chain reaction (PCR) reagents comprising three fluorescent compounds and wherein the first fluid and the second fluid are separated by a third fluid;
(d) imaging the defined flow path by illuminating the defined flow path using the illumination source and capturing three images of the defined flow path using the camera and the three camera filters, wherein one camera filter is positioned to filter light entering the camera when an image is captured, and subtracting the three background images from the three images to create an image set;
(e) registering the first fluid and the second fluid across the image set by calculating at least one fluid property of the first fluid and grouping the first fluid across the three images of the image set based on the at least one fluid property of the first fluid and calculating at least one fluid property of the second fluid and grouping the second fluid across the three images of the image set based on the at least one fluid property of the second fluid;
(f) calculating the fluorescent intensity of the first fluid and the second fluid in at least one image of the image set if the fluorescent intensity was not calculated in step (e) and optionally calculating at least one of the size and position of the first fluid and at least one of the size and position of the second fluid;
(g) controlling the temperature of the first fluid and the second fluid using the thermal device to amplify the at least one nucleic acid in the first fluid and the second fluid by polymerase chain reaction (PCR);
(h) imaging the defined flow path by illuminating the defined flow path using the illumination source and capturing three images of the defined flow path using the camera and the three camera filters, wherein one camera filter is positioned to filter light entering the camera when an image is captured, and subtracting the three background images from the three images to create an additional image set;
(i) registering the first fluid and the second fluid across the additional image set by calculating at least one fluid property of the first fluid and grouping the first fluid across the additional image set based on the at least one fluid property of the first fluid and calculating at least one fluid property of the second fluid and grouping the second fluid across the additional image set based on the at least one fluid property of the second fluid;
(j) calculating the fluorescent intensity of the first fluid and the second fluid in at least one image of the additional image set if the fluorescent intensity was not calculated in step (i) and optionally calculating at least one of the size and position of the first fluid and at least one of the size and position of the second fluid;
(k) repeating the sequence of steps (g), (h), (i), and (j) 10 to 40 times;
(l) computing the movement of the first fluid and the second fluid in the defined flow path and applying a correction factor for the movement;
(m) associating the fluorescent intensity of the first fluid and the second fluid in each image set with the at least one nucleic acid to create a fluorescent intensity group for each at least one nucleic acid for the first fluid and the second fluid;
(n) computing at least one curve property of the fluorescent intensity group for each at least one nucleic acid;
(o) comparing the at least one curve property to a library of the at least one curve property of known nucleic acids; and
(p) identifying the at least one nucleic acid in the sample.

19. The method for identifying at least one nucleic acid in a sample of claim 18, wherein step (g) controls the temperature of the first fluid and the second fluid by cycling the temperature of the first fluid and the second fluid between a temperature above 75 degrees Celsius (° C.) and a temperature below 75 degrees Celsius (° C.) using the thermal device.

20. The method for identifying at least one nucleic acid in a sample of claim 19, wherein the imager further comprises three illumination filters positionable to filter light exiting the illumination source and wherein one illumination filter is positioned to filter light exiting the illumination source when a background image or an image is captured.

21. The method for identifying at least one nucleic acid in a sample of claim 20, wherein the first fluid is aqueous, the second fluid is aqueous, and the third fluid is at least one of oil and gas.

22. The method for identifying at least one nucleic acid in a sample of claim 21, wherein the at least one nucleic acid is at least one of DNA and RNA.

23. The method for identifying at least one nucleic acid in a sample of claim 22, wherein the at least one nucleic acid is selected from the group consisting of viruses, bacteria, fungi, animals, insects, plants, and artificial constructs.

24. The method for identifying at least one nucleic acid in a sample of claim 22, wherein the at least one nucleic acid is selected from the group consisting of the species Escherichia coli, and the genera Streptococcus, Staphylococcus, Enterococcus, Klebsiella, Proteus, Acinetobacter, Pseudomonas, Enterobacter, Salmonella, Campylobacter, Chronobacter, and Listeria.

25. The method for identifying at least one nucleic acid in a sample of claim 22, wherein the at least one nucleic acid is selected from the group consisting of the genera Botrytis or Sclerotinia.
